(19) United States
(12) Patent Application Publication
(10) Pub. No.: US 2017/0333704 A1
(43) Pub. Date: Nov. 23, 2017

(54) LEADS FOR NEUROSTIMULATION AND METHOD OF ASSEMBLING THE SAME

(71) Applicant: Advanced Neuromodulation Systems, Inc., Plano, TX (US)

(72) Inventor: Aaron Raines, Dallas, TX (US)

(21) Appl. No.: 15/672,053

(22) Filed: Aug. 8, 2017

Related U.S. Application Data
(62) Division of application No. 13/898,715, filed on May 21, 2013, now abandoned.

(51) Int. Cl. A61N 1/08; A61N 1/05

(52) U.S. Cl. CPC A61N 1/08; A61N 1/0551; Y10T 29/49071; A61N 1/086

(57) ABSTRACT

A neurostimulator lead including an elongated lead body having stimulating and proximal end portions and a center axis extending therebetween. The lead body includes an inner tubing that extends along the center axis. The inner tubing includes wire conductors that extend between the stimulating and proximal end portions. The lead also includes multiple electrode-inductor assemblies that are positioned along the stimulating end portion and spaced apart from one another along the center axis. Each of the electrode-inductor assemblies includes an inductor coil that is electrically coupled to one of the wire conductors and an electrode that is located proximate to the inductor coil. The electrode and the inductor coil are electrically joined, and the inductor coil is configured to prevent a flow of induced current that occurs when the lead is exposed to external magnetic fields.

### CROSS REFERENCE TO RELATED APPLICATIONS

This application is a division of U.S. patent application Ser. No. 13/898,715, filed May 21, 2013.

### FIELD OF THE INVENTION

One or more embodiments of the subject matter described herein generally relate to systems having leads for generating electric fields proximate to anatomical tissue and, in more particular embodiments, for generating electric fields proximate to nerve tissue.

### BACKGROUND

Neurostimulation systems (NS) are devices that generate electrical pulses and deliver the pulses to nerve tissue to treat a variety of disorders. Spinal cord stimulation (SCS) is the most common type of neurostimulation. In SCS, electrical pulses are delivered to nerve tissue in the spine typically for the purpose of chronic pain control. While a precise understanding of the interaction between the applied electrical energy and the nerve tissue is not fully appreciated, it is known that application of an electric field to spinal nerve tissue can effectively mask or alleviate certain types of pain transmitted from regions of the body associated with the stimulated nerve tissue.

NS and SCS systems generally include a pulse generator and one or more leads electrically coupled to the pulse generator. A lead includes an elongated body of insulative material that encloses wire conductors. A stimulating end portion of the lead includes multiple electrodes that are electrically coupled to the wire conductors. The stimulating end portion of a lead is implanted proximate to nerve tissue (e.g., within epidural space of a spinal cord) to deliver the electrical pulses. A proximal end of the lead body includes multiple terminal contacts, which are also electrically coupled to the wire conductors. The terminal contacts, in turn, are electrically coupled to the pulse generator. The terminal contacts are configured to receive electrical pulses from the pulse generator that are then delivered to the electrodes through the wire conductors to generate the electric fields. The pulse generator is typically implanted within the individual and may be programmed (and re-programmed) to provide the electrical pulses in accordance with a designated sequence.

One challenge faced by designers of NS and SCS systems is that the systems may be prone to heating and induced current when placed in the strong static, gradient, and/or radiofrequency (RF) magnetic fields of a magnetic resonance imaging (MRI) system. The heat and induced current are the results of the leads acting as antennas in the magnetic fields generated during a MRI scan. The heat and induced current may result in deterioration of stimulation thresholds or, in the context of a cardiac lead, even increase the risk of cardiac tissue damage and perforation.

Yet many patients with an implantable pulse generator and an implanted lead may require, or at the very least can benefit from, a MRI scan in the diagnosis or treatment of a medical condition. MRI scans have even been proposed as a visualization mechanism for lead implantation procedures. As such, it is desirable to have NS systems that are MRI-compatible. To this end, known leads include inductor coils that are electrically coupled to the electrodes. The inductor coils are configured to prevent a flow of the induced current when the NS systems are exposed to different external magnetic fields.

The conventional leads include an elongated body that is formed from concentric inner and outer tubing. The wire conductors that join the electrodes and the inductor coils are located in an interior space between the inner and outer tubes. During manufacture, the wire conductors are inserted through the electrodes. However, the wire conductors are free-floating within the interior space and may also have relatively small diameters (e.g., less then microns). Accordingly, it may be difficult to capture and manipulate the wire conductors to join them to the electrode. The wire conductors are susceptible to breaking due to the small size. In addition, the electrical connections to the inductor coils (e.g., contacts and/or wires) and the wires of the inductor coils themselves may be small and, thus, difficult to manage and susceptible to breaking. Accordingly, the process of electrically joining the conductive components of the lead can be labor intensive and costly.

Therefore, a need remains for implantable leads and NS systems that are MRI-compatible and that are capable of being produced in a less costly manner than known leads and NS systems.

### BRIEF SUMMARY

In accordance with an embodiment, a neurostimulator lead is provided that includes an elongated lead body having stimulating and proximal end portions and a center axis extending therebetween. The lead body includes an inner tubing that extends along the center axis. The inner tubing includes wire conductors that extend between the stimulating and proximal end portions. The neurostimulator lead also includes multiple electrode-inductor assemblies that are positioned along the stimulating end portion and spaced apart from one another along the center axis. Each of the electrode-inductor assemblies includes an inductor coil that is electrically coupled to one of the wire conductors and an electrode that is located proximate to the inductor coil. The electrode and the inductor coil are electrically joined, and the inductor coil is configured to prevent a flow of induced current that occurs when the lead is exposed to external magnetic fields.

The electrode of at least one electrode-inductor assembly may have opposite exterior and interior surfaces. The exterior surface may be configured to interface with an individual to apply stimulating electrical pulses. The interior surface may face the inner tubing, wherein the inductor coil of the at least one electrode-inductor assembly may be located between the inner tubing and the interior surface of the electrode. In some embodiments, the at least one electrode-inductor assembly includes an electrical contact that is secured to the interior surface and that electrically joins the inductor coil and the electrode.

The electrode of at least one electrode-inductor assembly may constitute a ring electrode that defines an internal passage extending along the center axis. The inductor coil of the at least one electrode-inductor assembly may be disposed within the internal passage.

At least one electrode-inductor assembly may include an electrical contact that electrically couples the inductor coil and the electrode. For example, the inductor coil may include an insulative bobbin and a coil conductor that is wound about the insulative bobbin. The electrical contact may be secured to the insulative bobbin. As another example, the electrical contact may be directly connected to a coil conductor of the inductor coil and mechanically and electrically joined to the electrode.

The inner tubing may include an insulative material. The wire conductors may include embedded portions that extend along the center axis and are at least partially embedded within the insulative material. In some embodiments, the wire conductors may include terminating portions that are external to the inner tubing and are electrically coupled to the corresponding inductor coils. Optionally, the exposed terminating portions may extend at least one of away from the inner tubing or circumferentially along an outwardly-facing surface of the inner tubing.

In some embodiments, the inner tubing has channel recesses located along an outer surface of the Inner tubing. The channel recesses may have corresponding wire conductors disposed therein.

The electrode-inductor assemblies may include first and second electrode-inductor assemblies. The inductor coils of the first and second electrode-inductor assemblies may have at least one conductor that is wound into a designated number of layers in which each of the layers includes a designated number of turns. Optionally, the designated number of turns and layers are selected to define self-resonant frequencies of the inductor coils of the first and second electrode-inductor assemblies.

In another embodiment, a method of assembling a neurostimulator lead is provided. The method includes providing at least one electrode-inductor assembly including an inductor coil and a stimulating electrode that are electrically joined to each other. The inductor coil has a receiving passage and the electrode has an internal passage. The method also includes inserting an inner tubing through the receiving and internal passages. The inner tubing extends along a center axis and has a wire conductor extending therethrough. The wire conductor has a terminating portion. The method also includes electrically coupling the terminating portion of the wire conductor to the inductor coil. The electrode-inductor assembly is located at a stimulating end portion of the neurostimulator lead and is configured to prevent a flow of induced current that occurs when the lead is exposed to external magnetic fields.

Optionally, the electrically coupling operation may include removing the terminating portion from a channel recess of the inner tubing such that the terminating portion is exposed along an exterior of the inner tubing.

Other embodiments may include neurostimulating (NS) systems. The NS system may include, for example, one or more leads and a pulse generator that is operably coupled to the lead(s). The NS system may be configured to be implanted entirely within an individual (e.g., patient) or, alternatively, the lead may be implanted and the pulse generator may not be implanted.

While multiple embodiments are disclosed, still other embodiments of the described subject matter will become apparent to those skilled in the art from the following Detailed Description, which shows and describes illustrative embodiments of disclosed inventive subject matter. As will be realized, the inventive subject matter is capable of modifications in various aspects, all without departing from the spirit and scope of the described subject matter. Accordingly, the drawings and detailed description are to be regarded as illustrative in nature and not restrictive.

### BRIEF DESCRIPTION OF THE DRAWINGS

FIG. 1 is a perspective view of one embodiment of neurostimulating (NS) system in accordance with one embodiment.

FIG. 2 is an isolated view of a neurostimulator lead formed in accordance with one embodiment that may be used with the NS system of FIG. 1.

FIG. 3 is a perspective view of an exposed portion of the neurostimulator lead in accordance with one embodiment.

FIG. 4 illustrates a perspective view of a bobbin in accordance with one embodiment that may be used with the lead of FIG. 2.

FIG. 5 is a perspective view of the bobbin having electrical contacts coupled to an end of the bobbin in accordance with one embodiment.

FIG. 6 is a perspective view of an inductor coil formed in accordance with one embodiment that may include the bobbin of FIG. 4.

FIG. 7 is a perspective view of an electrode-inductor assembly in accordance with one embodiment.

FIG. 8 is an enlarged view of the lead with a portion of the lead removed to illustrate the electrode-inductor assembly in accordance with one embodiment.

FIG. 9 is an enlarged view of a distal end of the lead with a portion removed to illustrate the electrode-inductor assembly in accordance with one embodiment.

FIG. 10 is a perspective view of an exposed portion of the lead in accordance with one embodiment.

FIG. 11 is a flowchart illustrating a method of assembling a lead in accordance with one embodiment.

FIG. 12 illustrates different stages during the manufacture of a bobbin in accordance with one embodiment.

### DETAILED DESCRIPTION

One or more embodiments described herein provide a neurostimulation (NS) system and/or a lead of an NS system that are configured for stimulating tissue. The tissue may be nerve tissue, such as spinal cord tissue. However, in other embodiments, the tissue may be peripheral nerve tissue, deep brain tissue, cortical tissue, cardiac tissue, digestive tissue, pelvic floor tissue, or any other suitable tissue within the body of an individual (e.g., patient). The lead has a lead body with a stimulating end portion and a proximal end portion. The stimulating end portion includes electrodes that are configured to emit electrical pulses that generate electric fields proximate to the tissue. The proximal end portion is adapted to electrically couple to a pulse generator of the NS system. The pulse generator provides the electrical pulses to the neurostimulator lead. In particular embodiments, the NS system is implanted within the body of the patient and may be referred to as an implantable medical device (IMD).

One or more embodiments may include multiple inductor coils positioned axially along the stimulating end portion. The inductor coils may be electrically joined to corresponding electrodes along the stimulating end portion to form electrode-inductor assemblies. The inductor coils may be located proximate to the corresponding electrode. For example, in some embodiments, the inductor coil is disposed within a passage defined by the electrode such that the electrode surrounds the inductor coil. For some embodiments, when the inductor coil and the electrode are positioned proximate to each other, an electrical contact may extend between and electrically join the inductor coil and the electrode. The contact may have a thickness that renders the contact less susceptible to damage than known leads, which utilize thin wires for joining the electrodes and inductor coils.

The inductor coils may form inductive elements that prevent (e.g., attenuate, reduce, or impede) a flow of electric current that is induced in the lead when the lead is exposed to an external magnetic field, such as a magnetic field formed by a magnetic resonance imaging (MRI) system. By way of example, magnetic fields generated by MRI systems can be about 1.5 Tesla (T) or about 3.0 T. The inductor coils may have different coil configurations (e.g., different layers, different number of windings, etc.) that correlate to different self resonant frequencies (SRF) or ranges of SRF. Accordingly, at least some embodiments include neurostimulator leads that are MRI-compatible.

The leads include wire conductors that extend between the stimulating and proximal end portions. In some embodiments, the wire conductors may be at least partially embedded within an inner tubing of the lead body such that the wire conductors are secured to the inner tubing. The wire conductors may also include terminating portions that may be located external to the inner tubing and freely movable to electrically couple an inductor coil and/or an electrode. In particular embodiments, the terminating portions extend from embedded portions of the wire conductor at predetermined axial locations along the inner tubing. Accordingly, instead of wire conductors being freely movable throughout the lead body as in known leads, the wire conductors may have only limited segments (e.g., the terminating portions) that are accessible and movable during manufacture.

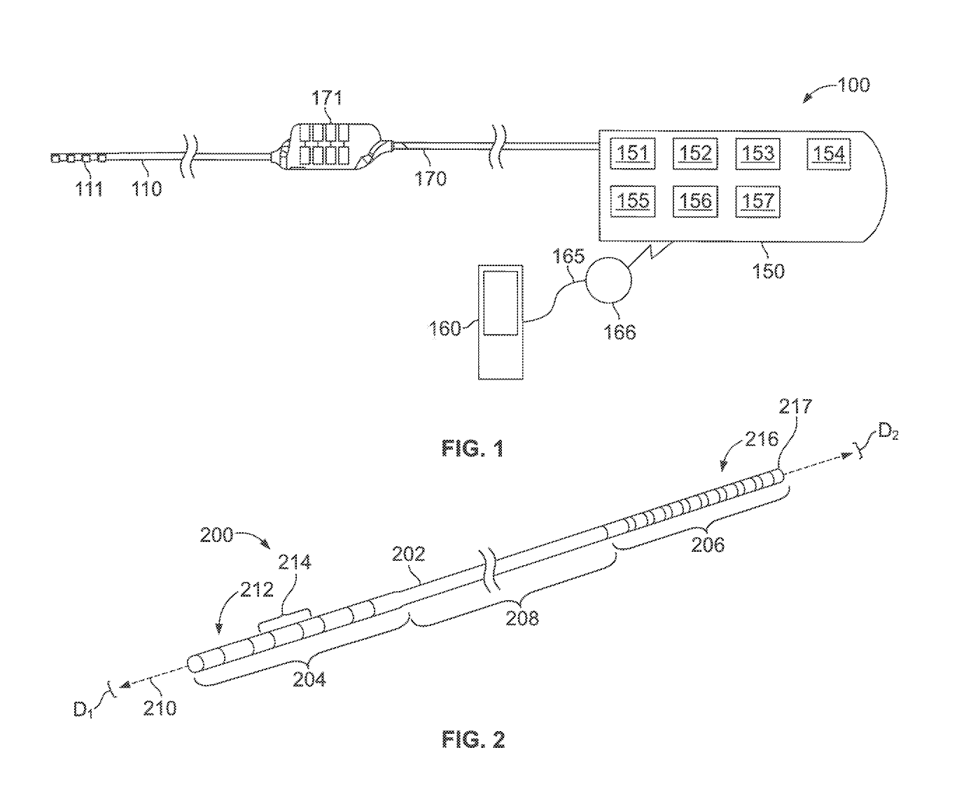

FIG. 1 depicts a neurostimulation (NS) system 100 that generates electrical pulses for application to tissue of a patient according to one embodiment. The tissue may be spinal cord tissue, peripheral nerve tissue, deep brain tissue, cortical tissue, cardiac tissue, digestive tissue, pelvic floor tissue, or any other suitable tissue within a body. For embodiments that stimulate spinal cord tissue, the nerve tissue may include dorsal column (DC) fibers and/or dorsal root (DR) fibers. The NS system 100 includes an NS device (or pulse generator) 150 that is adapted to generate electrical pulses in order to apply electric fields to the tissue. The NS device 150 is typically implantable within an individual (e.g., patient) and, as such, may be referred to as an implantable pulse generator (IPG). The implantable NS device 150 typically comprises a housing that encloses a controller 151, a pulse generating circuitry 152, a charging coil 153, a battery 154, a far-field and/or near field communication circuitry 155, a battery charging circuitry 156, a switching circuitry 157, etc. of the device. The controller 151 typically includes a processor or other logic-based device for controlling the various other components of the NS device 150. Software code is typically stored in memory of the NS device 150 for execution by the processor to control the various components of the device.

The NS device 150 may comprise a separate or an attached extension component 170. If the extension component 170 is a separate component, the extension component 170 may connect with the “header” portion of the NS device 150 as is known in the art. If the extension component 170 is integrated with the NS device 150, internal electrical connections may be made through respective conductive components. Within the NS device 150, electrical pulses are generated by the pulse generating circuitry 152 and are provided to the switching circuitry 157. The switching circuitry 157 connects to outputs of the NS device 150. Electrical connectors (e.g., “Bal-Seal” connectors) within the connector portion 171 of the extension component 170 or within the header may be employed to conduct the electrical pulses. Terminal contacts (not shown) of one or more neurostimulator leads 110 are inserted within the connector portion 171 or within the header for electrical connection with respective connectors. Thereby, the pulses originating from NS device 150 are provided to the neurostimulator lead 110. The pulses are then conducted through wire conductors of the lead 110 and applied to tissue of an Individual via electrodes 111.

For implementation of the components within NS device 150, a processor and associated charge control circuitry for an implantable pulse generator is described in U.S. Patent Publication No. 20060259098, entitled “SYSTEMS AND METHODS FOR USE IN PULSE GENERATION,” which is incorporated herein by reference. Circuitry for recharging a rechargeable battery of an implantable pulse generator using inductive coupling and external charging circuits are described in U.S. patent Ser. No. 11/109,114, entitled “IMPLANTABLE DEVICE AND SYSTEM FOR WIRELESS COMMUNICATION,” which is incorporated herein by reference.

An example and discussion of “constant current” pulse generating circuitry is provided in U.S. Patent Publication No. 20060170486 entitled “PULSE GENERATOR HAVING AN EFFICIENT FRACTIONAL VOLTAGE CONVERTER AND METHOD OF USE,” which is incorporated herein by reference. One or multiple sets of such circuitry may be provided within the NS device 150. Different pulses on different electrodes may be generated using a single set of pulse generating circuitry using consecutively generated pulses according to a “multi-stimset program.” Complex pulse parameters may be employed such as those described in U.S. Pat. No. 7,228,179, entitled “Method and apparatus for providing complex tissue stimulation patterns,” and International Patent Publication Number WO/2001/093953 A1, entitled “NEUROMODULATION THERAPY SYSTEM,” which are incorporated herein by reference. Alternatively, multiple sets of such circuitry may be employed to provide pulse patterns that include simultaneously generated and delivered stimulation pulses through various electrodes of one or more stimulation leads as is also known in the art. Various sets of parameters may define the pulse characteristics and pulse timing for the pulses applied to various electrodes as is known in the art. Although constant current pulse generating circuitry is contemplated for some embodiments, any other suitable type of pulse generating circuitry may be employed such as constant voltage pulse generating circuitry.

FIG. 2 is a perspective of a neurostimulator lead 200 formed in accordance with one embodiment that may be used as the lead 110 (FIG. 1) with the NS system 100 (FIG. 1). The lead 200 includes an elongated lead body 202 having a stimulating end portion 204 and a proximal end portion 206 and an intermediate segment 208 extending therebetween. The stimulating end portion 204 represents a portion of the lead 200 that provides electrical pulses that generate electric fields proximate to a designated location in an individual. The proximal end portion 206 represents a portion of the lead 200 that operably couples with an NS device, such as the NS device 150. For instance, the proximal end portion 206 receives the electrical pulses from the NS device and transmits the electrical pulses to the stimulating end portion 204 through the intermediate segment 208.

A center axis 210 of the lead 200 extends through the lead body 202 between the stimulating and proximal end portions 204, 206. Although the lead body 202 appears linear in FIG. 2, the lead body 202 may be permitted to flex or bend. As such, the center axis 210 may twist and turn with the lead body 202 into different configurations. The center axis 210 generally overlaps with or corresponds to a center of a cross-section of the lead body 202 taken at any point transverse to the elongated lead body 202.

The stimulating end portion 204 includes an array of electrodes (or contacts) 212 that are axially spaced apart from one another along the center axis 210. More specifically, the electrodes 212 may have designated axial locations along the stimulating end portion 204. Adjacent electrodes 212 have a center-to-center spacing 214 therebetween. The center-to-center spacing 214 may be configured, along with other features of the NS system, to provide electric fields with designated characteristics when the pulses are applied. For example, the center-to-center spacing 214 between two electrodes 212 may be at least about 40 mils. The center-to-center spacing 214 between the different electrodes may be equal throughout the stimulating end portion or, alternatively, vary such that one electrode may be located at different distances from the two electrodes that are adjacent to the one electrode.

The proximal end portion 206 includes an array of terminal contacts 216 that are axially spaced apart from one another along the center axis 210. The proximal end portion 206 may also include an end cap 217. Each of the terminal contacts 216 is configured to engage a corresponding contact of the NS system. In the illustrated embodiment, the lead 200 includes four electrodes 212 and eight terminal contacts 216. The lead 200 may also include one or more inactive “dummy” contacts along the proximal end portion 206. However, embodiments are not limited to the arrangements of electrodes 212 and terminal contacts 216 shown in FIG. 2 and other configurations may be used.

In some embodiments, the lead 200 may be characterized as a percutaneous lead that is configured to be inserted into the individual through a needle (e.g., spinal needle). For example, the stimulating end portion 204 may have a diameter D₁ of about 2.0 mm or less. In the illustrated embodiment, the diameter D₁ of the stimulating end portion 204 is greater than a diameter D₂ of the proximal end portion 206. However, the diameters D₁ and D₂ may be equal to each other in other embodiments, or the diameter D₂ may be greater than the diameter D₁.

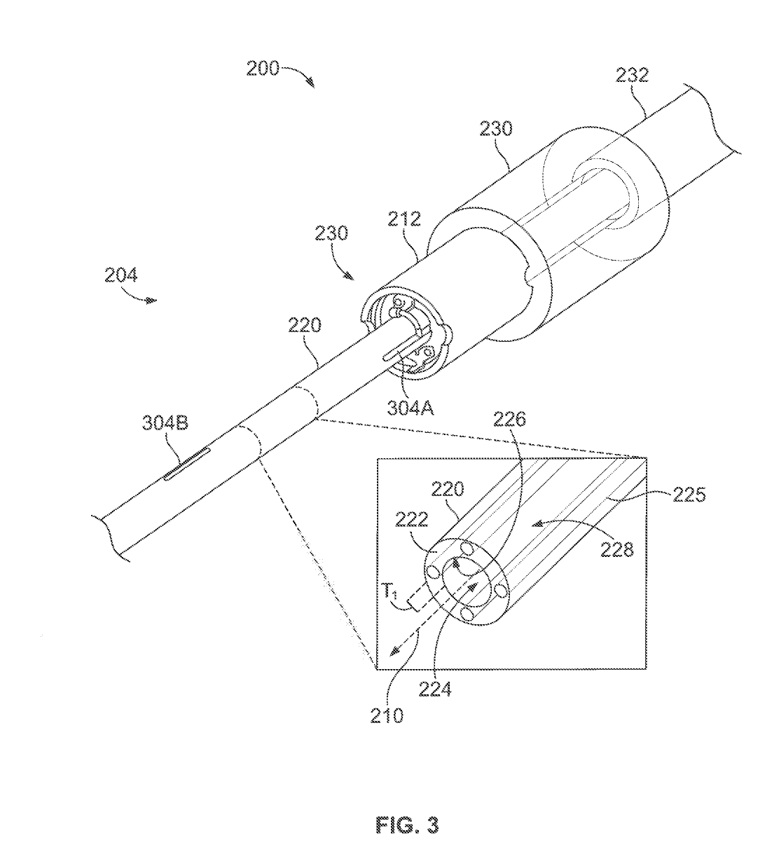

FIG. 3 is a perspective view of an exposed portion of the neurostimulator lead 200. In particular, an incomplete section of the stimulating end portion 204 is shown. The lead 200 includes an inner tubing 220. The inner tubing 220 may comprise a non-metallic or insulative material, such as a polymer. The material may be a flexible or elastic material that allows the inner tubing 220 to bend or be shaped in a designated manner. As shown in the enlarged phantom portion of the inner tubing 220, the inner tubing 220 may have a channel surface 226 that faces radially inward, an outer surface 228 that faces radially outward, and a tube wall 222 that extends radially between the channel and outer surfaces 226, 228. A thickness T₁ of the tube wall 222 is defined between the channel and outer surfaces 226, 228. The channel surface 226 defines a longitudinal channel 224 of the inner tubing 220 that extends along the center axis 210.

In the illustrated embodiment, the inner tubing 220 includes the channel 224. However, in other embodiments, the inner tubing 220 may not have the channel 224 or the inner tubing 220 may have portions that do not include the channel 224. More specifically, the inner tubing 220 may include portions that are completely solid and do not include the channel 224. For some embodiments, the channel 224 may receive a rod or stylet that is approximately equal to a cross-sectional area of the channel 224. The rod may stiffen the lead 200 thereby enabling a doctor (or other suitably qualified person) to insert the lead 200 into the individual.

The inner tubing 220 includes a plurality of wire conductors 225 that are at least partially embedded within the inner tubing 220. For example, in FIG. 3, the wire conductors 225 are completely embedded within the tube wall 222 such that the non-metallic material of the inner tubing 220 completely surrounds the wire conductors 225. In other embodiments, however, the tube wall 222 may only partially embed the wire conductors 225 such that a longitudinal portion of the wire conductor 225 is exposed along at least one of the outer surface 228 or the channel surface 226. In the illustrated embodiment, the tube wall 222 is formed from only the insulative material and the wire conductors 225. For example, the insulative material may be molded or extruded over the wire conductors 225. In alternative embodiments, the tube wall 222 may include multiple layers with similar or different materials.

As shown, the wire conductors 225 may extend in a linear manner such that the wire conductors extend parallel to the center axis 210. However, in other embodiments, the wire conductors 225 may pitch in a helical manner such that the wire conductors 225 twist or wrap about the center axis 210. The linear configuration may provide more tensile strength to the lead 200, whereas the helical configuration may allow the lead 200 to bend more readily.

The lead 200 includes an electrode-inductor assembly 230 having at least one of the electrodes 212, an outer tubing 232 that surrounds the inner tubing 220, and a spacer 234 that is located between the outer tubing 232 and the electrode-inductor assembly 230. The outer tubing 232 may include a channel (not shown) having a diameter that is greater than a diameter of the inner tubing 220. The inner tubing 220 can be inserted into the channel of the outer tubing 232, or the outer tubing 232 may be formed around the inner tubing 220. Alternatively, the inner tubing 220 and the outer tubing 232 may be one single tubing that has a varying diameter.

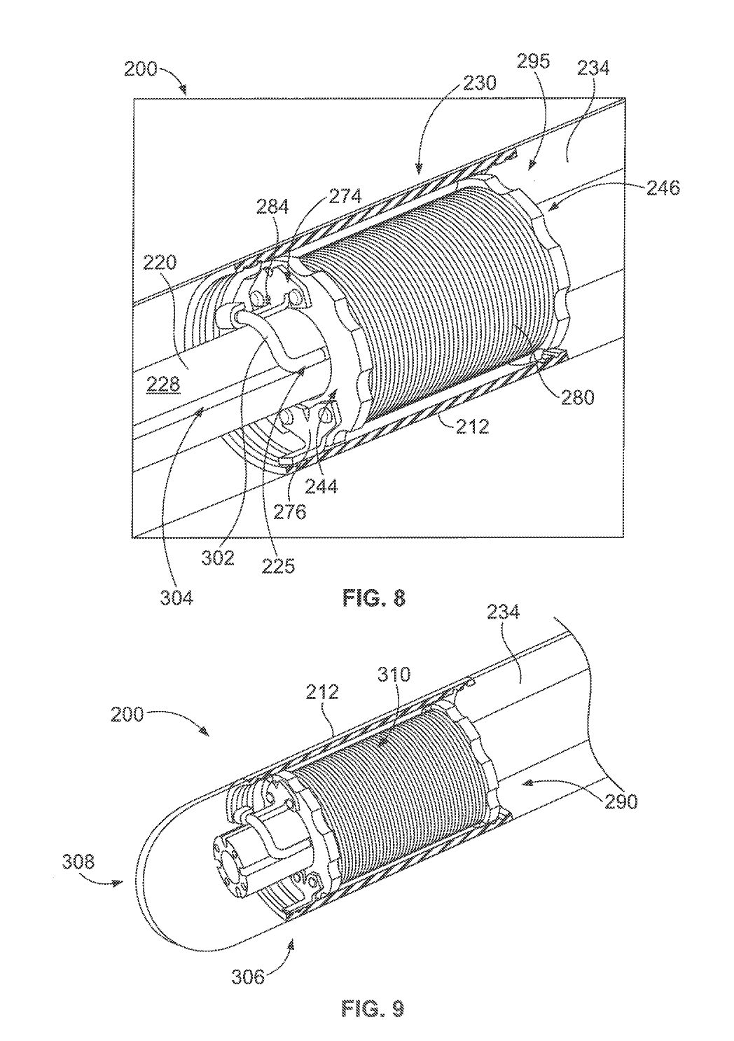

Although only a single electrode-inductor assembly 230 and a single spacer 234 are shown in FIG. 3, the inner tubing 220 is configured to receive multiple electrode-inductor assemblies 230 and spacers 234 along the stimulating end portion 204. During assembly, the electrode-inductor assemblies 230 and the spacers 234 may be positioned at predetermined axial locations. As described herein, when the electrode-inductor assemblies 230 are positioned at the predetermined axial locations the electrode-inductor assemblies 230 may be proximate to terminating portions 302 (shown in FIG. 8) of the wire conductors 225. The terminating portions 302 of the wire conductors 225 may then be electrically coupled to the electrode-inductor assemblies 230.

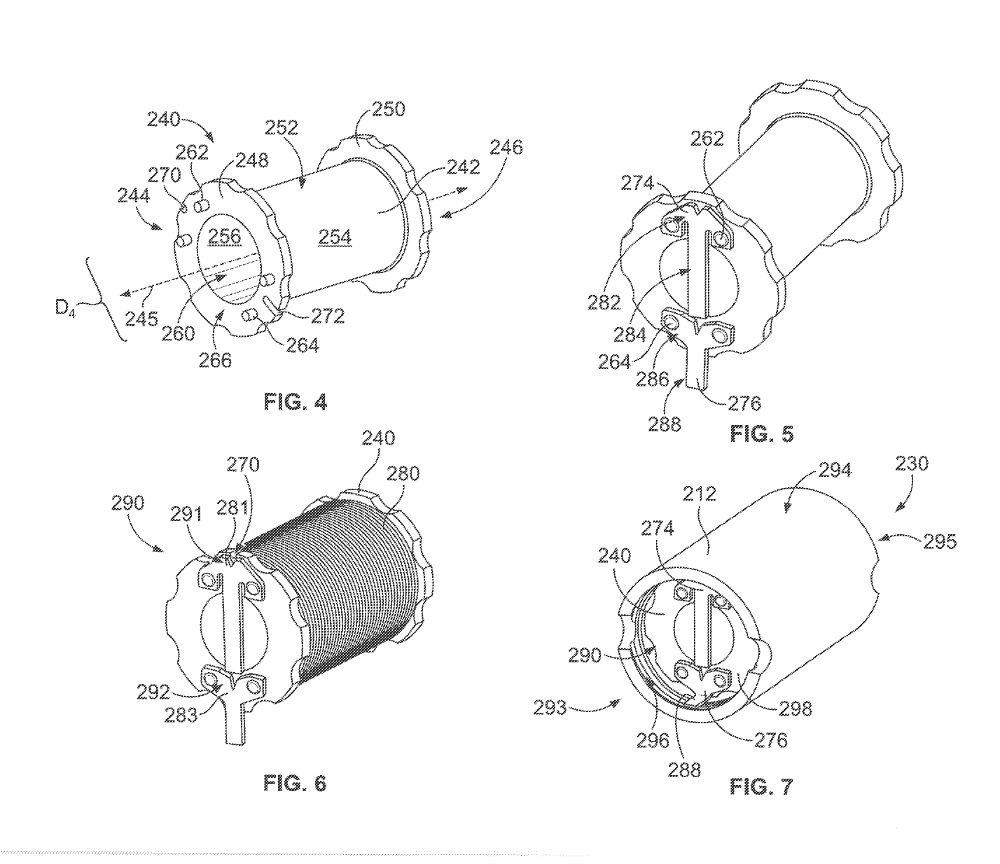

FIGS. 4-7 illustrate different stages of assembly for one of the electrode-inductor assemblies 230 (shown in FIG. 7) in accordance with one embodiment. FIGS. 4-7 illustrate, however, just one method of assembling the electrode-inductor assembly 230 and other methods may be used. Moreover, the lead 200 (FIG. 2) may have one or more electrode-inductor assemblies 230 that are assembled in the manner described herein and one or more electrode-inductor assemblies that are assembled in a different manner.

FIG. 4 shows an isolated perspective view of a bobbin 240 that is configured to have a coil conductor 280 (shown in FIG. 6) wound about the bobbin 240. The bobbin 240 includes a support body 242 having opposite bobbin ends 244, 246. In the illustrated embodiment, the support body 242 of the bobbin 240 extends completely around (e.g., circumferentially around) a central longitudinal axis 245 that extends between the bobbin ends 244, 246. When the lead 200 is fully assembled, the longitudinal axis 245 and the center axis 210 (FIG. 2) may substantially coincide.

The bobbin 240 includes opposite end walls 248, 250 located proximate to the bobbin ends 244, 246, respectively, and a coil-supporting section 252 that extends longitudinally between the bobbin ends 244, 246. The bobbin 240 has an outer surface 254 and an interior or inner surface 256 that extend circumferentially around the longitudinal axis 245. The outer surface 254 faces radially away from the longitudinal axis 245. In particular embodiments, the end walls 248, 250 project radially outward from the coil-supporting section 252 such that a cylindrical space is defined between the end walls 248, 250. In other embodiments, only one of the end walls 248, 250 may project radially outward from the coil-supporting section 252. Alternatively, the end walls 248, 250 may not project radially outward and, instead, may be substantially flush with the coil-supporting section 252.

The inner surface 256 defines a receiving passage 260 that extends between the ends 244, 246. A cross-section of the receiving passage 260 taken transverse to the longitudinal axis 245 may be sized and shaped to receive the inner tubing 220 (FIG. 3). For example, the cross-section of the receiving passage 260 may be circular and have a diameter D₄ that is greater than the diameter D₃ of the inner tubing 220.

The bobbin end 244 may include contact-securing features 262, 264. In the illustrated embodiment, the contact-securing features 262, 264 are posts that project in a direction parallel to the longitudinal axis 245. More specifically, the end wall 248 may have an end face 266 that faces in a direction parallel to the longitudinal axis 245 and includes the contact-securing features 262, 264. The embodiment shown in FIG. 4 includes two contact-securing features 262 and two contact-securing features 264. In other embodiments, there may be fewer or more contact-securing features 262, 264. Also, in alternative embodiments, one or more of the contact-securing features 262, 264 may be positioned proximate to the bobbin end 246.

The bobbin 240 may also include conductor-receiving slots 270, 272 that are sized and shaped to receive the coil conductor 280 (FIG. 6). As shown, each of the conductor-receiving slots 270, 272 may extend in a radial direction along the end wall 248. In other embodiments, the conductor-receiving slot 270 may be located within the end wall 248, and the conductor-receiving slot 272 may be located within the opposite end wall 250.

FIG. 5 is an isolated perspective view of the bobbin 240 having electrical contacts 274, 276 secured thereto. The electrical contact 274 is coupled to the contact-securing features 262, and the electrical contact 276 is coupled to the contact-securing features 264. The electrical contacts 274, 276 may include holes (not shown) that receive the contact-securing features 262, 264. The contact-securing features 262, 264 may then be molded or heated so that the contact-securing features 262, 264 secure the corresponding electrical contact to the bobbin 240. The electrical contacts 274, 276, however, may be secured to the bobbin 240 using other methods. For example, the electrical contacts 274, 276 may be coupled to the contact-securing features 262, 264 through an interference fit alone. The electrical contacts 274, 276 may also be staked into the support body 242.

The electrical contact 274 includes a body portion 282 and a coupling portion 284. The electrical contact 276 includes a body portion 286 and a coupling portion 288. In the illustrated embodiment, the body portions 282, 286 are configured to directly engage the bobbin 204 when the electrical contacts 274, 276 are secured to the bobbin 240. In the illustrated embodiment, the coupling portions 284, 288 are shaped as tails or tabs. In FIG. 5, the coupling portions 284, 288 have not been deflected (e.g., bent or shaped) and extend in a common direction that is orthogonal (e.g., perpendicular) to the longitudinal axis 245 (FIG. 4). When the lead 200 is fully assembled, the coupling portions 284, 288 may extend generally along the longitudinal axis 245.

FIG. 6 is a perspective view of an inductor coil 290. The inductor coil 290 includes the bobbin 240 having the coil conductor 280 wound about the bobbin 240 and, more particularly, about the coil-supporting section 252 (FIG. 4). The inductor coil 290 may form an inductive element that prevents the flow of induced current in the lead 200 when the lead 200 is exposed to an external magnetic field, such as a magnetic field formed by a magnetic resonance imaging (MRI) system.

The inductor coils 290 of different electrode-inductor assemblies 230 along the stimulating end portion 204 may have different coil or winding configurations that correlate to different self resonant frequencies (SRFs) or ranges of SRFs. For example, the coil conductor 280 of the corresponding inductor coil 290 may be wound into a designated number of layers in which each of the layers includes a designated number of turns. The designated number of turns and layers may be selected to define a SRF of the inductor coil 290. In some embodiments, one coil conductor 290 of an inductor coil 290 may have layers with a greater (or fewer) number of turns than another conductor coil 290 of a different inductor coil 290. For instance, a first inductor coil may have a SRF range that includes 64 megahertz and a second inductor coil may have a SRF range that includes 128 megahertz. The first and second inductor coils may be adjacent or non-adjacent to each other along the stimulating end portion.

The number of turns can represent the number of times that the coil conductor 280 of the respective inductor coil 290 is wrapped around the bobbin 240 or the longitudinal axis 245 within a unit length. Alternatively, the number of turns can represent the total number of times that the coil conductor 280 is wrapped around the bobbin 240 or the longitudinal axis 245. The number of layers represents the number of concentric layers formed by the coil conductors 280.

The inductor coils 290 may have one or more layers with one or more turns. Non-limiting examples of such inductor coils include an inductor coil having 2 layers of turns with 10 turns per layer; an inductor coil having 3 layers of turns with 20 turns per layer, an inductor coil having 4 layers of turns with 6 turns per layer; and an inductor coil having 5 layers of turns with 30 turns per layer. Moreover, the different layers of a single inductor coil may have a different number of turns. By way of a specific example, the coil conductor of one inductor coil may have outer and inner layers with 14 turns and a middle layer with 19 turns. A different inductor coil may have outer and inner layers with 12 turns and a middle layer with 14 turns. The above winding configurations are just examples and the coil conductor 280 (or the inductor coil 290) may have other winding configurations.

Additional options for tuning the inductor coil may include increasing or decreasing a wire diameter of the coil conductor 280 and/or a pitch distance (e.g., distance between common points of adjacent turns measured along the longitudinal axis 245) may be modified.

Accordingly, one or more parameters of the conductive pathways (e.g., coil conductors) may be varied or changed to adjust the SRF of the corresponding inductor coil. For example, changing a thickness or gauge of the coil conductor, a thickness and/or type of material used for the insulation surrounding the coil conductor, the number of turns that the coil conductor is wrapped in a coil, the pitch distance, and/or the number of layers of turns of the coil conductor may be varied or changed to tune or set the SRF of the coil conductor.

The winding configuration of the coil conductor 280 causes the coil conductor 280 to form an electrically inductive element in a circuit that includes the electrode-inductor assembly 230 (FIG. 3). For example, when exposed to a relatively strong external magnetic field, such as a magnetic field generated by an MRI system, the inductor coil 290 may have a relatively large electrical impedance characteristic at a designated frequency (e.g., about 64 or about 128 Mhz). The impedance characteristic can prevent the electric current that is induced by the magnetic field of the MRI system from flowing to the electrode 212 (FIG. 3). The impedance characteristic may increase when the inductor coil 290 is exposed to an external magnetic field having a frequency that is equal to or approximately equal to the SRF of the inductor coil 290 or is within a range of SRFs of the inductor coil 290. As a result, the SRF of the winding configuration of the coil conductor 280 causes the impedance of the inductor coil 290 to increase when exposed to an external magnetic field having the same or similar frequency as the associated SRF. One or more aspects of the winding configuration can be changed to adjust the SRF.

As shown in FIG. 6, the electrical contacts 274, 276 include conductor-receiving slots 291, 292. For example, the body portions 282, 286 include the conductor-receiving slots 291, 292, respectively. The conductor-receiving slot 291 of the electrical contact 274 is configured to overlap or align with the conductor-receiving slot 270 of the bobbin 240. The conductor-receiving slot 292 of the electrical contact 276 is configured to overlap or align with the conductor-receiving slot 272 (FIG. 4) of the bobbin 240. The coil conductor 280 includes two conductor ends 281, 283. The conductor end 281 is inserted through the conductor-receiving slots 291, 270, and the conductor end 283 may be inserted through the conductor-receiving slots 292, 272. The conductor ends 281, 283 may be coupled to the electrical contacts 274, 276, respectively. For example, the conductor ends 281, 283 may be soldered or laser-welded to the electrical contacts 274, 276, respectively.

FIG. 7 is a perspective view of an assembled electrode-inductor assembly 230 in accordance with one embodiment. The electrode-inductor assembly 230 includes the inductor coil 290 and one of the electrodes 212. The inductor coil 290 and the electrode 212 are electrically joined to the inductor coil 290. The electrode 212 has opposite exterior and interior surfaces 294, 296 and extends between opposite electrode ends 293, 295. The exterior surface 294 is configured to interface with the individual to apply stimulating electric fields. The interior surface 296 is configured to face the inner tubing 220 (FIG. 3).

The electrode 212 may be located proximate to the inductor coil 290. For example, the Interior surface 296 of the electrode 212 may define an internal passage 298 that extends around and along the longitudinal axis 245 (FIG. 4) of the bobbin 240. The inductor coil 290 may be disposed within (e.g., partially or entirely within) the internal passage 298. In the Illustrated embodiment, the electrode 212 constitutes a ring electrode that extends around the inductor coil 290. The electrode 212 may completely surround the inductor coil 290 such that the inductor coil 290 does not clear the ends 293, 295 of the electrode 212.

The electrical contacts 274, 276 are configured to electrically couple the electrode 212 to one of the wire conductors 225 (FIG. 3) in the inner tubing 220 (FIG. 3). In the illustrated embodiment, the electrical contact 274 is configured to electrically couple to the wire conductor 225, and the electrical contact 276 is configured to electrically couple to the electrode 212. As shown in FIG. 7, the electrical contact 276 is coupled to the interior surface 296 of the electrode 212. In some embodiments, the electrical contact 276 is directly connected to the interior surface 296. For example, the coupling portion 288 of the electrical contact 276 may be soldered or welded to the interior surface 296.

In particular embodiments, as the bobbin 240 is inserted into the internal passage 298 through the electrode end 293, the coupling portion 288 may directly engage the electrode 212. For example, when the bobbin 240 is almost entirely within the electrode 212, the coupling portion 288 is deflected radially inward toward the longitudinal axis 245. The deflected coupling portion 288 may slide along the interior surface 296 until the bobbin 240 is positioned at the designated location with respect to the electrode 212. The coupling portion 288 may then be mechanically and electrically coupled to the electrode 212 through, for example, soldering or welding.

In other embodiments, however, the electrode 212 and the inductor coil 290 may only partially overlap each other such that at least a portion of the inductor coil 290 clears one of the ends 293, 295 along the longitudinal axis 245. In some embodiments, the inductor coil 290 and the electrode 212 may not overlap. Instead, the inductor coil 290 and the electrode 212 may be axially offset with respect to each other along the center axis 210 by a separation distance. In such embodiments, the electrical contact 276 may be configured to extend across the separation distance to electrically join the inductor coil 290 and the electrode 212.

FIG. 8 is an enlarged view of the lead 200 with a portion of the lead 200 removed to illustrate one of the electrode-inductor assemblies 230 after the lead 200 has been manufactured. FIG. 8 shows the lead 200 after the lead 200 has undergone a molding or reflow operation in which the polymer material of the spacer 234 has been melted about and within the electrode-inductor assemblies 230 and the inner tubing 220.

Prior to the molding operation, the electrode-inductor assemblies 230 may be loaded onto the inner tubing 220. The loading operation may be similar to loading beads onto a thread to form a necklace. For example, the inner tubing 220 may be inserted through the electrode end 295 and through the bobbin end 246 and into the receiving passage 260 (FIG. 4). When the inner tubing 220 is proximate to the bobbin end 244 and begins to clear the receiving passage 260, the inner tubing 220 may engage the coupling portion 284 of the electrical contact 274 and deflect the coupling portion 284 radially away from the center axis 210 (FIG. 2). The coupling portion 284 may slide along the outer surface 228 of the inner tubing 220 until the electrode-inductor assembly 230 is positioned at a designated axial location.

The designated axial location for each electrode-inductor assembly 230 may be relative to a terminating portion 302 of the wire conductor 225. The terminating portion 302 of the wire conductor 225 may represent the portion of the wire conductor 225 that is external to the inner tubing 220 and is configured to couple to the electrode-inductor assembly 230. The terminating portion 302 may be shaped to extend along the outer surface 228.

In the illustrated embodiment, the inner tubing 220 includes a channel recess 304. The channel recess 304 may be formed removing the insulative material of the inner tubing 220. The material may be removed by, for example, chemically or optically ablating the material so that the material proximate to and along the terminating portion 302 of the wire conductor 225 is removed. More specifically, the wire conductor 225 may be at least partially embedded before the ablating operation and, after the ablating operation, may be removable from the channel recess 304. In some embodiments, the ablating and/or removal operations may include cutting wire conductor 225 to form the terminating portion 302.

Once the terminating portion 302 is removed from the channel recess 304, the terminating portion 302 may be electrically coupled to the coupling portion 284 of the electrical contact 274. For example, as shown in FIG. 8, the terminating portion 302 may be removed such that the terminating portion 302 clears the channel recess 304 and is then wrapped about the center axis 210 along the outer surface 228.

The coupling portion 284 may be directly coupled to the terminating portion 302. For example, the coupling portion 284 may be shaped to wrap around and grip the terminating portion 302. The coupling portion 284 may then be laser-welded to the terminating portion 302. Other methods of terminating a wire conductor to an electrical contact may be used (e.g., soldering, ultrasonic welding, and the like).

Accordingly, after the inductor coil 290 is coupled to the electrode 212 and the wire conductor 225, a conductive pathway may exist through the lead 200. The conductive pathway may extend along the inner tubing 220 through the wire conductor 225, through the electrical contact 274 to the inductor coil 290, through the inductor coil 290 to the electrical contact 276, and through the electrical contact 276 to the electrode 212. The conductive pathway may include fewer electrical connections (e.g., terminations) than the conductive pathways of other known neurostimulator leads. For example, the conductive pathway of the Illustrated embodiment includes only four electrical connections: (1) the connection between the electrode 212 and the electrical contact 276; (2) the connection between the electrical contact 276 and the coil conductor 280; (3) the connection between the coil conductor 280 and the electrical contact 274; and (4) the connection between the electrical contact 274 and the wire conductor 225.

Moreover, the conductive pathway may be more resilient than the conductive pathways of other known neurostimulator leads. For instance, the wire conductors 225 are not free floating. Instead, the wire conductors 225 may be at least partially embedded within the inner tubing 220 and, thus, protected, by the insulative material of the inner tubing 220. In addition, the electrical contacts 274, 276 may be dimensioned to withstand movement of the lead 200. For example, the dimensions of the electrical contacts may be greater than the dimensions of connecting wires used by known neurostimulator leads. In addition, more resilient methods of termination (e.g., soldering or various types of welding) may be used to form the electrical connections.

However, embodiments of the claimed subject matter are not required to have each and every feature described herein. For example, alternative embodiments may include the wire conductors 225 being at least partially embedded within the inner tubing 220, but may not include the electrical contacts 274, 276 as shown in FIG. 8. Embodiments may include the configuration of the electrode-inductor assembly 230 as shown in FIG. 8, but may not include the wire conductors being embedded within the inner tubing 220.

In alternative embodiments, the terminating portions 302 of the wire conductors 225 are directly coupled to the coil conductor 280. For example, the terminating portion 302 may configured to extend from the inner tubing 220 and toward or over one of the end walls 248, 250 (FIG. 4). The terminating portion 302 may be directly coupled to one of the conductor ends through, for example, laser welding.

Returning to FIG. 3, adjacent channel recesses 304 may be circumferentially offset with respect to one another. For example, FIG. 3 shows channel recesses 304A and 304B. The channel recess 304A may be located at, for example, 3 o'clock with respect to the center axis 210, but the channel recess 304B may be located at 12 o'clock with respect to the center axis 210. The electrode-inductor assemblies 230 that engage the wire conductors 225 associated with the channel recesses 304A, 304B may be positioned in different manners for coupling to the wire conductors 225. For example, the electrode-inductor assemblies 230 that are positioned proximate to the channel recesses 304A, 304B may have different rotational orientations with respect to the inner tubing 220.

FIG. 9 is an enlarged view of the lead 200. The lead 200 includes a leading cap 308 at a distal end 306 of the lead 200. The leading cap 308 may be formed during the molding operation from a spacer 234 that is positioned at the distal end 306. For example, the material of the spacers 234 on either side of the electrode-inductor assemblies 230 may seep into voids and cavities that exist in the lead 200 when the spacers 234 are melted. As one example, the material of the spacer 234 may flow into a void space 310 that is located between the electrode 212 and the inductor coil 290. The leading cap 308 may be shaped for insertion into body of the patient. After the molding operation, the exterior surface 294 of the electrode 212 may have insulative material from the spacer 234. Optionally, the material along the exterior surface 294 may be removed through, for example, ablation or stripping.

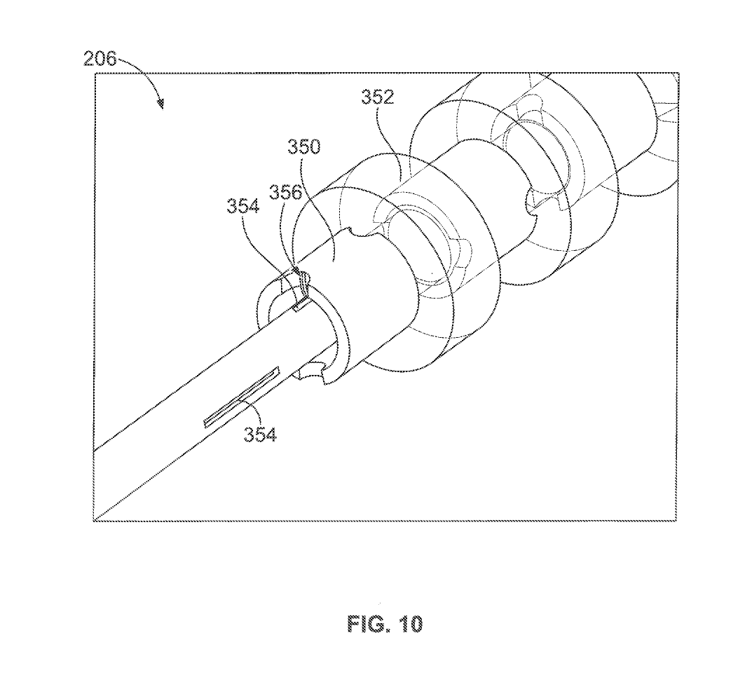

FIG. 10 is a perspective view of an exposed portion of an Incomplete section of the proximal end portion 206. The proximal end portion 206 may be manufactured in a similar manner as described above with respect to the stimulating end portion 204 of the lead 200. For example, the proximal end portion 206 may include terminal contacts 350, which may be identical or similar to the electrodes 212 (FIG. 2). The terminal contacts 350 may be positioned at different axial locations with spacers 352 located between adjacent terminal contacts 350.

In particular embodiments, the terminal contacts 350 may be positioned proximate to channel recesses 354. Similar to the channel recesses 304A, 304B (FIG. 3), adjacent channel recesses 354 may be circumferentially offset with respect to each other. The wire conductors 225 (FIG. 3) may have terminating portions 356 at the proximal end portion 206 that are disposed within the channel recesses 354. The terminating portions 356 may be withdrawn from the corresponding channel recesses 354 and coupled to the terminal contacts 350. For example, the terminating portion 356 in FIG. 10 may be welded (e.g., laser-welded) to the corresponding terminal contact 350. Similar to the stimulating end portion 204, the proximal end portion 206 may be formed during a molding operation. During the molding operation, melted material from the spacers 352 may flow into voids and spaces along the proximal end portion 206. The stimulating and proximal end portions 204, 206 may undergo the respective molding operations simultaneously or at separate times.

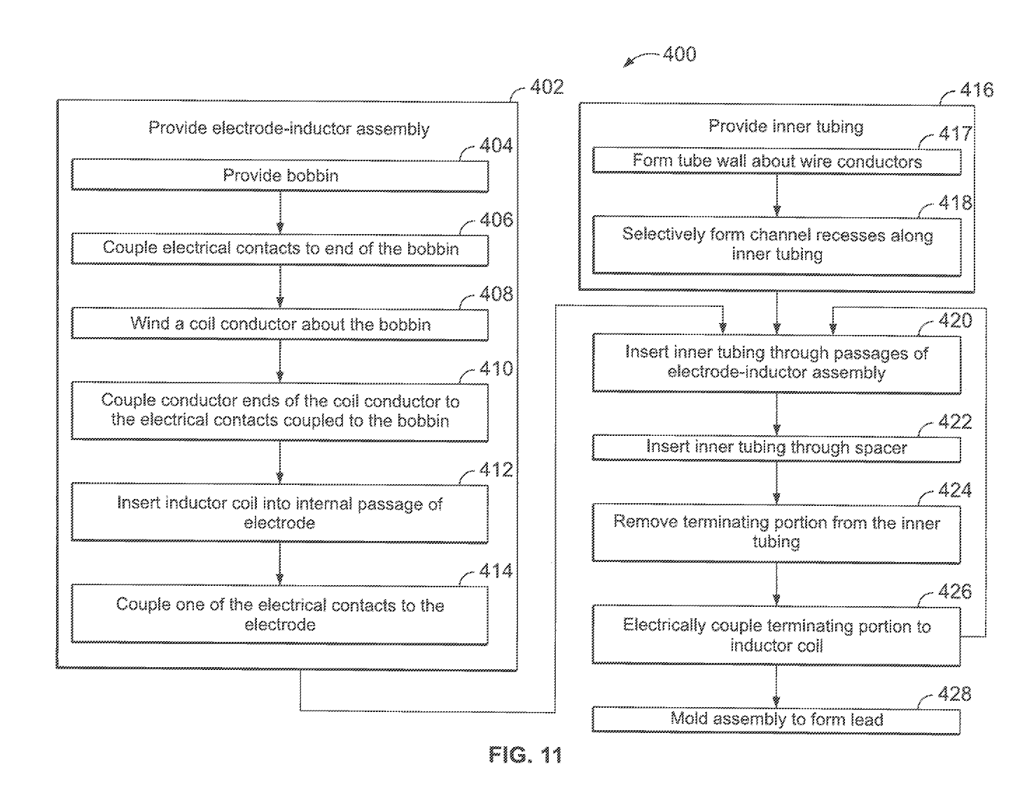

FIG. 11 is a flowchart illustrating a method 400 of assembling a lead in accordance with one embodiment. The method 400, for example, may employ structures or aspects of various embodiments discussed herein, such as those described with respect to FIGS. 1-10. In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion.

At 402, the method 400 may include providing at least one electrode-inductor assembly that has an inductor coil and a stimulating electrode electrically joined to the inductor coil. The electrode-inductor assembly may be similar to the electrode-inductor assembly 230 (FIG. 3) described herein. The providing operation (at 402) may include providing a pre-constructed electrode-inductor assembly or may include at least partially constructing the electrode-inductor assembly. For example, the providing operation (at 402) may include providing (at 404) a bobbin for forming the electrode-inductor assembly. The providing operation (at 402) may also include a coupling operation (at 406) in which electrical contacts are coupled to the bobbin.

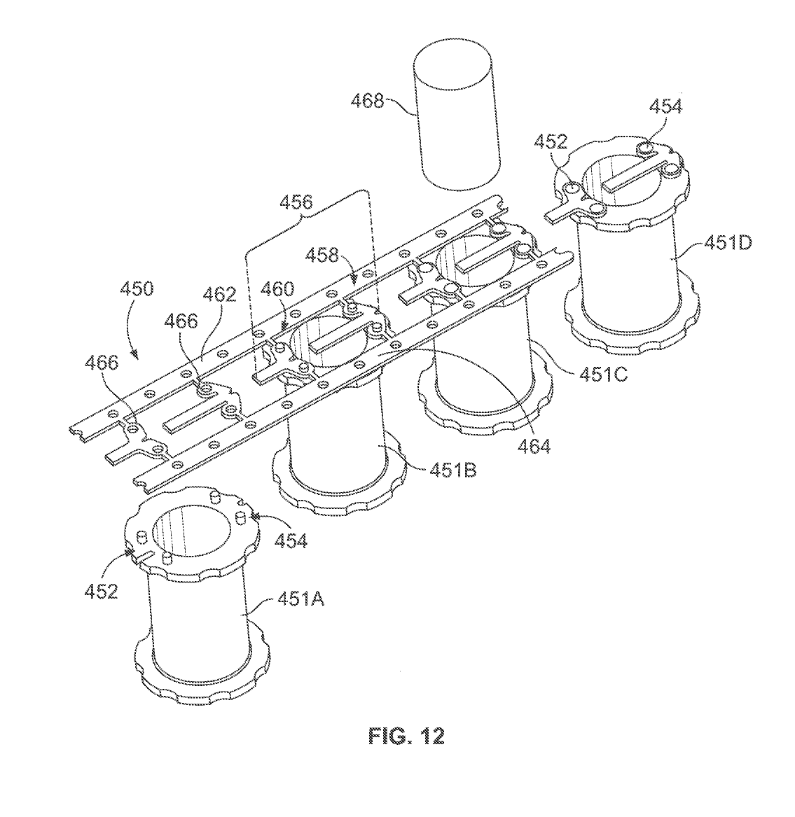

FIG. 12 illustrates the coupling operation (at 406). As shown, a contact ribbon 450 and a plurality of bobbins 451A-451D may be provided. Each of the bobbins 451A-451D has contact-securing features 452, 454, which are illustrated as posts or projections in FIG. 12. The contact ribbon 450 constitutes a sheet of material that was stamped to provide a series of contact pairs 456 having electrical contacts 458, 460 adjacent to each other in each pair. Each of the electrical contacts 458, 460 is secured between two rails 462, 464 of the contact ribbon 450. The electrical contacts 458, 460 may be identical or similar to the electrical contacts 274, 276, respectively, shown in FIG. 5.

The coupling operation (at 406) includes securing each contact pair 456 to one of the corresponding bobbins 451A-451D. As shown, the contact-securing features 452, 454 may be inserted through holes 466 of the electrical contacts 458, 460. The contact-securing features 452, 454 may form a frictional engagement to the electrical contacts 458, 460 (e.g., interference fit). In some embodiments, an adhesive may be used to couple the electrical contacts 458, 460 to the corresponding bobbin 451. In alternative embodiments, the contact-securing features 452, 454 may be cavities that receive and frictionally engage portions of the electrical contacts 458, 460. Other methods of coupling the electrical contacts 458, 460 to the corresponding bobbin may be used.

Optionally, the coupling operation (at 406) may include heating the contact-securing features 452, 454 using a thermal element 468 to melt the contact-securing feature 452, 454 and then allowing the contact-securing feature 452, 454 to solidify (e.g., cure or harden). As shown with the bobbin 451D, the bobbin 451D may be separated from the rails 462, 464 through, for example, a stamping operation or laser-ablation.

Returning to FIG. 11, the method 400 may also include winding (at 408) a coil conductor about the bobbin thereby forming an inductor coil. The inductor coil may be tuned for a designated SRF by winding a designated number of coil layers about the bobbin in which each coil layer has a designated number of turns. In an exemplary embodiment, the coil conductor has opposite conductor ends that are each directly coupled (at 410) to one of the electrical contacts. At 412, the inductor coil may be inserted into an internal passage of an electrode. One of the electrical contacts may be coupled (at 414) to the electrode. For example, as described above, the electrical contact may engage and be deflected by the electrode during the inserting operation (at 412). The electrical contact may then be directly coupled to an interior surface of the electrode through, for example, welding.

At 416, an inner tubing, such as the inner tubing 220 (FIG. 3), may be provided. The inner tubing 220 may be previously formed or the method 400 may include forming the inner tubing. For example, the method 400 may include forming (at 417) a tube wall about a plurality of wire conductors such that the wire conductors are at least partially embedded within the material of the tube wall. The providing operation (at 416) may also include selectively forming (at 418) channel recesses along an outer surface of the inner tubing. The forming operation (at 418) may include removing material of the inner tubing such that wire conductors are exposed within the channel recesses. The channel recesses may have designated axial locations. The channel recesses may be axially spaced apart by a predetermined separation distance. In some embodiments, the channel recesses may be circumferentially offset with respect to each other.

At 420, the inner tubing may be inserted through the receiving and internal passages of the electrode-inductor assembly. In some embodiments, the inner tubing is also inserted through (at 422) a central passage of a spacer that is configured to be positioned between adjacent electrode-inductor assemblies along the inner tubing. Each of the electrode-inductor assemblies may be positioned at a predetermined axial location that is proximate to one of the channel recesses.

Terminating portions of the wire conductors may be removed (at 424) such that the terminating portion is exposed along an exterior of the inner tubing. In some embodiments, the terminating portions may be wrapped along an outer surface of the inner tubing to engage the electrical contact. The terminating portions may then be electrically coupled (at 426) to the inductor coils of the electrode-inductor assemblies. By way of one example, a tool may be inserted into each of the channel recesses to remove (at 424) the corresponding terminating portion of the wire conductor. The terminating portion may then be shaped (e.g., manipulated by the tool) to engage the electrical contact. In particular embodiments, the terminating portion is gripped by the electrical contact and the electrical contact and the terminating portion are welded together thereby electrically coupling the inductor coil and the wire conductor.

The method 400 may include repeating the operations at 420, 422, 424, and 426 until a predetermined number of electrode-inductor assemblies are located along the inner tubing. At 428, the inner tubing having the electrode-inductor assemblies and the spacers thereon may be molded so that longitudinal portions have a substantially uniform cross-section. For example, the assembly may be molded to form the lead 200 shown in FIG. 2. In some embodiments, the molding operation includes the application of heat and pressure to the assembly of electrode-inductor assemblies and spacers. In some embodiments, the molding operation includes positioning the inner tubing having the electrode-inductor assemblies thereon into a preformed mold and apply heat and/or pressure.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings. Also, it is to be understood that phraseology and terminology used herein with reference to device or element orientation (such as, for example, terms like “central,” “upper,” “lower,” “front,” “rear,” “distal,” “proximal,” and the like) are only used to simplify description of one or more embodiments described herein, and do not alone indicate or imply that the device or element referred to must have a particular orientation. In addition, terms such as “outer” and “inner” are used herein for purposes of description and are not intended to indicate or imply relative importance or significance.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the presently described subject matter without departing from its scope. While the dimensions, types of materials and coatings described herein are intended to define the parameters of the disclosed subject matter, they are by no means limiting and are exemplary embodiments. Many other embodiments will be apparent to those of skill in the art upon reviewing the above description. The scope of the inventive subject matter should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

Although the invention has been described with reference to certain embodiments, persons skilled in the art will recognize that changes may be made in form and detail without departing from the spirit and scope of the invention.

What is claimed is:

1. A method of assembling a neurostimulator lead, the method comprising:
providing at least one electrode-inductor assembly including an inductor coil and a stimulating electrode that are electrically joined to each other, the inductor coil having a receiving passage and the electrode having an internal passage;
inserting an inner tubing through the receiving and internal passages, the inner tubing extending along a center axis and having a wire conductor extending therethrough, the wire conductor having a terminating portion; and
electrically coupling the terminating portion of the wire conductor to the inductor coil, wherein the electrode-inductor assembly is located at a stimulating end portion of the lead and is configured to prevent a flow of induced current that occurs when the lead is exposed to external magnetic fields.

2. The method of claim 1, wherein the electrically coupling operation includes removing the terminating portion from a channel recess of the inner tubing such that the terminating portion is exposed along an exterior of the inner tubing.

3. The method of claim 2, further comprising removing material of the inner tubing to define the channel recess and expose the terminating portion.

4. The method of claim 1, wherein the electrically coupling operation includes circumferentially wrapping the terminating portion about the inner tubing.

5. The method of claim 1, wherein the wire conductor is a first wire conductor and the inner tubing comprises a second wire conductor having a terminating portion, the terminating portions of the first and second wire conductors being held in fixed positions along the inner tubing, the fixed positions having different axial locations along the center axis.

6. The method of claim 1, wherein the electrode of at least one electrode-inductor assembly has opposite exterior and interior surfaces, the exterior surface configured to interface with an individual to apply stimulating electrical pulses, the interior surface facing the inner tubing, wherein the inductor coil of the at least one electrode-inductor assembly is located between the inner tubing and the interior surface of the electrode.

7. The method of claim 1, wherein the electrode of at least one electrode-inductor assembly constitutes a ring electrode that defines a receiving passage extending along the center axis, the inductor coil of the at least one electrode-inductor assembly being disposed within the receiving passage.

8. The method of claim 1, wherein the at least one electrode-inductor assembly includes an electrical contact that is electrically coupled to the inductor coil, wherein the electrically coupling operation includes mechanically and electrical coupling the terminating portion of the wire conductor to the electrical contact.
